(19) United States
(12) Patent Application Publication
(10) Pub. No.: US 2006/0235372 A1
(43) Pub. Date: Oct. 19, 2006

(54) Facilitating tools for cardiac tissue ablation

(76) Inventor: Jim L. Ward, Laguna Niguel, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/399,111

(22) Filed: Apr. 5, 2006

Related U.S. Application Data
(60) Provisional application No. 60/668,663, filed on Apr. 6, 2005.

(51) Int. Cl. A61B 18/18

(52) U.S. Cl. 606/13

(57) ABSTRACT

Methods and devices are provided for positioning a device around the pulmonary veins of a heart to treat atrial fibrillation. One method includes the steps of guiding a first elongate member through the transverse sinus, guiding a second elongate member through the oblique sinus, connecting a distal end of the first elongate member to a distal end of the second elongate member, and removing one of the first and second elongate members to position the other of the first and second elongate members around the pulmonary veins. In another exemplary embodiment, a method is disclosed that includes a step of guiding a elongate member through the transverse sinus superior to the right superior pulmonary vein and the left superior pulmonary vein of a patient's heart, and then along the pericardial sac wall and through the pericardial reflection inferior to the left inferior pulmonary vein and the right inferior pulmonary vein to extend out of the patient's heart such that the elongate member encircles the pulmonary veins. Ablative energy can then be delivered to form a lesion encircling the pulmonary veins. Kits of facilitating tools and ablation instruments are also disclosed.

### CROSS REFERENCE TO A RELATED PATENT APPLICATION

The present application claims priority to U.S. Provisional Patent Application No. 60/668,663 filed on Apr. 6, 2005, the disclosure of which is incorporated by reference herein in its entirety.

### FIELD OF THE INVENTION

The technical field of the invention is surgery and, in particular, methods and devices for facilitating placement of ablation devices on tissue.

### BACKGROUND OF THE INVENTION

Atrial fibrillation (AF) is a form of cardiac arrhythmia characterized by rapid randomized contractions of atrial myocardium, causing an irregular, often rapid ventricular rate. The regular pumping function of the atria is replaced by a disorganized, ineffective quivering. Atrial fibrillation is caused, in large part, by chaotic conduction of electrical signals through the upper chambers of the heart. Atrial fibrillation is often associated with other forms of cardiovascular disease, including congestive heart failure, rheumatic heart disease, coronary artery disease, left ventricular hypertrophy, cardiomyopathy or hypertension.

Various techniques have been proposed for the treatment of atrial fibrillation by blocking errant electrical signal conduction in the heart. For example, the “Maze procedure” was developed in the early 1990s to treat atrial fibrillation by forming a series of physical incisions (in the form of a maze) to create scar tissue in the heart that would stop the electrical impulses. Although these procedures were originally performed with a scalpel, various other techniques have also been developed to form lesions.

Researchers have come to realize that the origins of AF often lie in the left atrium in the vicinity of the pulmonary veins. A lesion encircling the pulmonary veins is currently considered to be most effective as a block. Several types of ablation devices have been proposed for encircling the pulmonary veins, however one problem with these devices is placement. Surgical ablation devices used to create lesions on cardiac atrial tissue must be correctly located to ensure effective conduction block and to prevent damage to tissues not intended to be treated. Current surgical practice is to use standard cardiac instruments to place an ablation device on the heart. In addition, some surgeons resort to the use of sutures, umbilical tape, non-cardiac catheters, and the like in an attempt to successfully place their devices. Consequently, surgical ablation procedures tend to be slow. Minimally invasive approaches can be even more problematic.

Accordingly, there remains a need for improved methods and devices for treating atrial fibrillation, and in particular for methods and devices to facilitate positioning of an ablation instrument around the pulmonary veins.

### SUMMARY OF THE INVENTION

The present invention provides methods and devices that facilitate placement of cardiac ablation tools and, in an exemplary embodiment, placement of an epicardial ablation device around the pulmonary veins. In one embodiment, a method for positioning a device around the pulmonary veins of a heart is provided and includes guiding a first elongate member through the transverse sinus, guiding a second elongate member through the oblique sinus, and connecting a distal end of the first elongate member to a distal end of the second elongate member. One of the first and second elongate members can then be retracted to position the other of the first and second elongate members around the pulmonary veins. In an exemplary embodiment, the first elongate member is guided through the transverse sinus by positioning the first elongate member superior to the right superior pulmonary vein and the left superior pulmonary vein, and the second elongate member is guided through the oblique sinus by positioning the second elongate member inferior to the right inferior pulmonary vein and the left inferior pulmonary vein. More particularly, the first elongate member can be introduced into the transverse sinus between the superior vena cava and the right superior pulmonary vein, and the second elongate member can be introduced into the oblique sinus between the inferior vena cava and the right inferior pulmonary vein. In a further embodiment, the first elongate member can be guided through the transverse sinus by positioning a distal end of the first elongate member adjacent to the pericardial sac, and the second elongate member can be guided through the oblique sinus by positioning a distal end of the second elongate member adjacent to the pericardial sac.

In the exemplary embodiment, the method can further include the step of inserting an ablation instrument through either the first or the second elongate member (once it has been positioned in an encircling configuration), and activating the ablation instrument to deliver ablative energy to form a lesion encircling the pulmonary veins. The ablation instrument can be activated while sliding the ablation instrument through the first elongate member as ablative energy is delivered to form a lesion encircling the pulmonary veins. In one embodiment, ablative energy can be delivered as a beam of energy substantially transverse to a longitudinal axis of the ablation instrument to selectively ablate the epicardial tissue and avoid damage to surrounding tissue.

In another exemplary embodiment, a method for treating atrial fibrillation is provided and includes guiding a elongate member through the transverse sinus superior to the right superior pulmonary vein and the left superior pulmonary vein of a patient's heart. The elongate member can have a distal tip that is adapted to guide the elongate member along the pericardial sac wall and through the pericardial reflection inferior to the left inferior pulmonary vein and the right inferior pulmonary vein to extend out of the patient's heart such that the elongate member encircles the pulmonary veins. Ablative energy can then be delivered (e.g., by an energy emitting element inserted into the elongate member following placement around the heart) to form a lesion encircling the pulmonary veins.

While various techniques can be used to guide the elongate member through the transverse sinus, in one embodiment the elongate member can be introduced through an incision formed in the pericardial reflection between the superior vena cava and the right superior pulmonary vein, and the elongate member can be inserted through the incision. A second incision can be formed in the pericardial reflection between the inferior vena cava and the right inferior pulmonary vein, and the elongate member can exit through the second incision.

A variety of techniques can also be used to deliver ablative energy to the tissue, but in one exemplary embodiment ablative energy is delivered by inserting an ablation instrument through the elongate member, and activating the ablation instrument to deliver ablative energy. The ablation instrument can be slid through the elongate member while delivering ablative energy. In another embodiment, the ablation instrument can be coupled to a proximal end of the elongate member and the elongate member can be pulled to position the ablation element around the pulmonary veins. Exemplary sources of ablative energy include optical radiation, microwaves, ultrasound, radio frequency, electrical current, and cryoablative sources.

In yet another embodiment of the present invention, a kit for treating atrial fibrillation is provided and includes one or more elongate members having flexible proximal and distal portions that are adapted to bluntly guide the elongate member through the transverse sinus, along the pericardial sac wall, and through the oblique sinus to encircle the pulmonary veins, and an ablation instrument adapted to couple to the elongate member and to deliver ablative energy to form a lesion encircling the pulmonary veins. The distal portions of each elongate member can have a variety of configurations, but in one embodiment the distal portion of at least one of the elongate members is curved. In another embodiment, the elongate member includes a wire extending therethrough and adapted to provide stiffness and shape to the distal portion. The ablation element can also have a variety of configurations, but in one embodiment it can be slidably disposable through the elongate member. In another embodiment, the ablation instrument can include a distal end that is adapted to couple to a proximal end of the elongate member. In yet another embodiment, the kit can include several elongate members having distal portions that differ relative to one another. In other aspects, the kit can include a tool having a grasping element formed on a distal end thereof and adapted to grasp a distal end of the elongate member to pull the elongate member through the oblique sinus.

### BRIEF DESCRIPTION OF THE DRAWINGS

FIG. 1A is a side, partially cross-sectional view of one embodiment of a facilitating tool including a pair of elongate members for use in positioning an ablation device;

FIG. 1B is a cross-sectional view of a human heart showing the facilitating tool of FIG. 1A about to be positioned around the pulmonary veins;

FIG. 2A is a side view of a first elongate member having a guide wire extending therefrom in accordance with another exemplary embodiment of a facilitating tool for use in positioning an ablation device;

FIG. 2B is a cross-sectional view of a human heart showing the first elongate member of FIG. 2A and a grasping member about to be positioned around the pulmonary veins;

FIG. 3A is a side view of a first elongate member having a curved distal end in accordance with yet another exemplary embodiment of a facilitating tool for use in positioning an ablation device;

FIG. 3B is a cross-sectional view of a human heart showing the first elongate member of FIG. 3A about to be positioned around the pulmonary veins;

FIG. 4A illustrates one exemplary embodiment of a distal tip portion for use with a facilitating tool in accordance with the present invention; and

FIG. 4B illustrates another exemplary embodiment of a distal tip portion for use with a facilitating tool in accordance with the present invention.

### DETAILED DESCRIPTION OF THE INVENTION

The present invention provides various methods and devices for positioning an epicardial ablation device on tissue, and more preferably for safely and effectively positioning an ablation device around the pulmonary veins of a heart. In an exemplary embodiment, a facilitating tool is provided and it is adapted to be guided around the pulmonary veins to encircle the veins. An ablation device can then be passed through the tool to ablation tissue, or the tool can be used to pull and position the ablation device around the veins. The tool is particularly useful in minimally invasive cardiac surgery because the tool can include features to guide the tool around the pulmonary veins which are located within the pericardium and thus are not visually accessible by the surgeon. A person skilled in the art will appreciate that, while the methods and devices are described for use in treating atrial fibrillation, the methods and devices can be used to perform a variety of other surgical procedures.

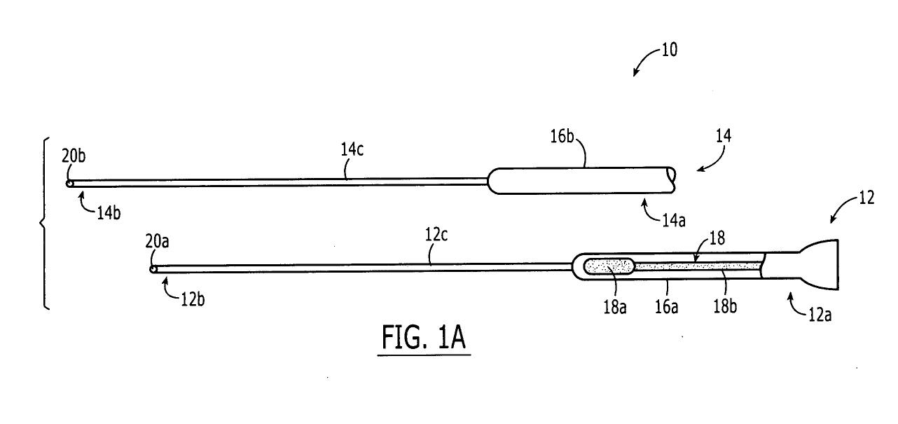

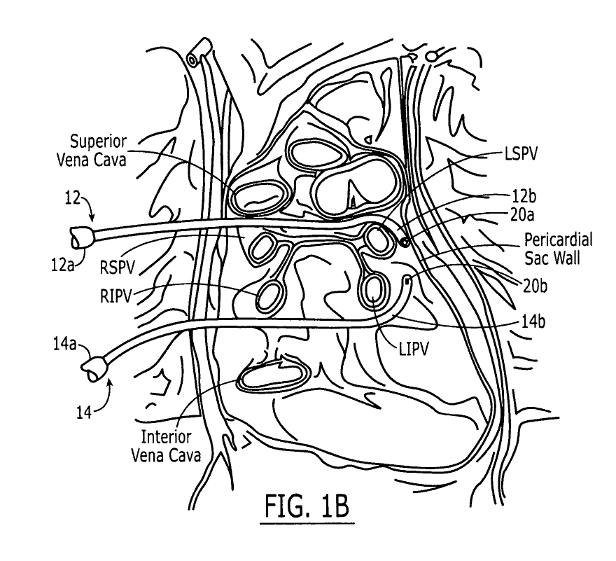

FIG. 1A illustrates one exemplary embodiment of a facilitating tool for positioning an ablation device, for example around the pulmonary veins. In this embodiment, the tool 10 generally includes first and second catheters 12, 14 that are adapted to be guided through the heart to encircle the pulmonary veins. A distal end of one or both catheters 12, 14 can be adapted to couple to one another to allow one of the catheters to be used to pull the other catheter around the pulmonary veins. Once positioned around the pulmonary veins, an ablation device can be slidably disposed through one of the catheters, or it can be attached to one of the catheters and the catheter can be used to pull the ablation device and position it around the pulmonary veins.

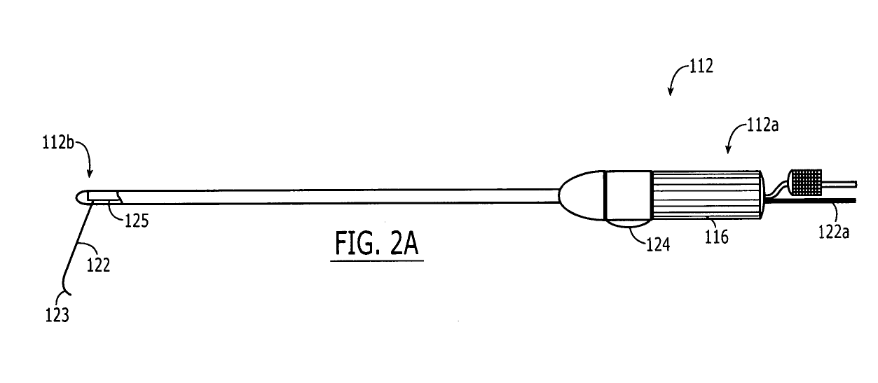

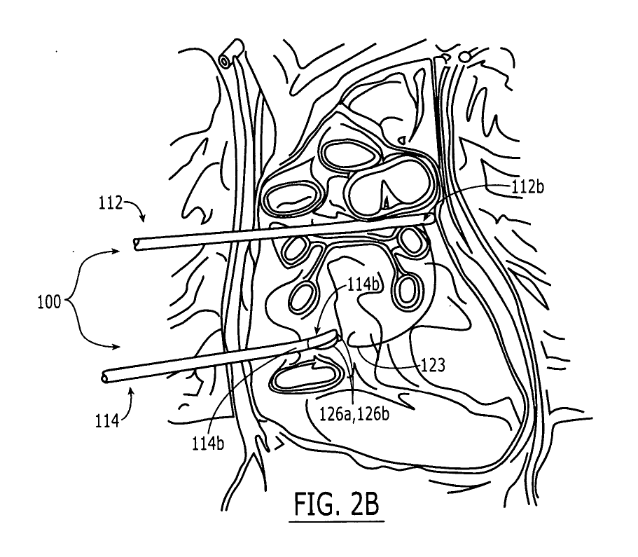

Each catheter 12, 14 can have a variety of configurations, but in the illustrated embodiment each catheter 12, 14 has a generally elongate shape with an inner lumen 12c, 14c extending between a proximal end 12a, 14a and a distal end 12b, 14b. The body of each catheter 12, 14 is preferably substantially flexible to allow the catheters 12, 14 to be easily guided through the heart and positioned around the pulmonary veins, however a person skilled in the art will appreciate that the particular properties of the catheters 12, 14 can be adjusted to facilitate positioning. The proximal end 12a, 14a of each catheter 12, 14 can also have a variety of configurations, but as shown in FIG. 1A the proximal end 12a, 14a of each catheter 12, 14 has a handle 16a, 16b formed thereon to facilitate grasping and manipulation of the catheters 12, 14. In another embodiment, the proximal end 12a, 14a of one or both catheters 12, 14 can include or be adapted to receive an ablation element therethrough, as will be discussed in more detail below. The distal end 12b, 14b of each catheter 12, 14 can also have a variety of configurations, but in an exemplary embodiment at least one of the distal ends 12b, 14b includes a mating or grasping element formed thereon and adapted to engage the distal end 12b, 14b of the other catheter 12, 14. FIG. 1A illustrates a magnet 20a, 20b disposed within the distal end 12b, 14b of each catheter 12, 14, such that the distal end 12b, 14b of the catheters 12, 14 will magnetically engage one another. Other exemplary mating or grasping elements, some of which will be discussed in more detail below with reference to FIGS. 2A-2B, include hooks, clamps, magnets, tube couplings, sutures, Velcro®, etc. A person skilled in the art will appreciate that a variety of techniques can be used to provide a removably matable connection between the first and second catheters 12, 14.

FIG. 1B illustrates facilitating tool 10 in use. As indicated above, in an exemplary embodiment the facilitating tool 10 is adapted for use in positioning an ablation device around the pulmonary veins of a heart. The exemplary procedure preferably begins by forming a superior dissection through the pericardial reflection between the superior vena cava and the right superior pulmonary vein into the interatrial groove. The superior dissection will provide access to the transverse sinus, which provides a path leading into the oblique sinus. An inferior dissection is then preferably formed through the pericardial reflection between the inferior vena cava and the right inferior pulmonary vein. The inferior dissection will provide access to the oblique sinus. A person skilled in the art will appreciate that, while FIG. 1B illustrates a cross-sectional view of the heart showing the pulmonary veins, an exemplary procedure in accordance with the present invention may be performed without direct visualization of the pulmonary veins, as the pulmonary veins are surrounded by the pericardium, which may only be dissected minimally to provide surgical access to the heart.

Once this dual path access has been established, the first catheter 12 can be guided through the transverse sinus and the second catheter 14 can be guided through the oblique sinus, as shown. In particular, the first catheter 12 can be introduced between the superior vena cava and the right superior pulmonary vein (RSPV) and guided through the transverse sinus at a position superior to the right and left superior pulmonary veins (RSPV, LSPV). The distal end 12b of the first catheter 12 will eventually come into contact with the pericardial sac wall, which will force the first catheter 12 in an inferior direction. The second catheter 12 can be introduced between the inferior vena cava and the right inferior pulmonary vein (RIPV), and guided through the oblique sinus at a position inferior to the right and left inferior pulmonary veins (RIPV, LIPV). The distal end 14b of the second catheter 14 will eventually come into contact with the pericardial sac wall, which will force the second catheter 14 in a superior direction. A person skilled in the art will appreciate that the first and second catheters 12, 14 can be formed from certain materials and can have certain properties that will facilitate movement and positioning of the catheters 12, 14 around the pulmonary veins. As the two catheters 12, 14 come toward one another, the mating elements, e.g., the magnets 20a, 20b, will mate with one another to mate the distal end 12b, 14b of each catheter 12, 14. One of the catheters, e.g., the second catheter 14, can then be pulled to pull the first catheter 12 completely around the pulmonary veins.

Once the first catheter 12 is positioned around the pulmonary veins, an ablation device can be introduced through the first catheter 12 to form a lesion around the pulmonary veins. FIG. 1A illustrates an ablation element 18 having a diffusion tip 18a formed on a distal end of an elongate catheter 18b. The diffusion tip 18a will direct ablative energy passing through the catheter toward tissue, thereby forming an ablation lesion around the pulmonary veins. Depending on the particular configuration of the ablation device, the device can be slidably moved through the first catheter 12 as energy is delivered to form an encircling lesion. Ablation element 18 is described in more detail in U.S. patent application Ser. No. 10/756,660 filed on Jan. 13, 2004 and entitled “Surgical Ablation System with Sliding Ablation Device,” which is hereby incorporated by reference in its entirety.

In another embodiment, an ablation device can be coupled to the proximal end 12a of the first catheter 12 and the first catheter 12 can be further pulled to pull and position the ablation device around the pulmonary veins. As previously discussed, FIG. 1A illustrates one exemplary embodiment of an ablation device 18 that is slidably disposed through the first catheter 12. While not shown in FIG. 1B, the ablation device 18 can be passed through the lumen 12c in the first catheter 12 and activated to form an encircling lesion around the pulmonary veins.

FIGS. 2A-2B illustrate another exemplary embodiment of a facilitating tool 100 for positioning an ablation device. As shown, the tool 100 includes first and second members 112, 114, each having a generally elongate shape with a proximal end (only the proximal end 112a of the first member 112 is shown) and a distal end 112b, 114b. In this embodiment, the first member 112 includes a handle 116 formed on the proximal end 112a and a guide wire 122 extending from the distal end 112b, and the second member 114 includes a grasping element formed on the distal end 114b thereof for grasping the guide wire 122 to mate the first and second members 112, 114.

The guide wire 122 of the first member 112 can have a variety of configurations, but in an exemplary embodiment the guide wire 122 is generally elongate and includes a hook-shaped distal tip 123. In use, the guide wire 122 is adapted to be disposed within the first member 112 and slidably movable relative to the first member 112. In particular, the guide wire 122 can be movable between a retracted position, in which the guide wire 122 is fully disposed within the first member 112 to allow the first member 112 to be inserted through tissue, and a second extended position, in which the guide wire 122 extends distally from the first member 112. In an exemplary embodiment, the guide wire 122 extends through a side opening 125 formed in the distal end 112b of the first member 112 such that the guide wire 122 extends in a direction that is substantially transverse to a longitudinal axis of the first member 112 to allow the guide wire 122 to extend toward the second member 114, as will be discussed in more detail below.

Various techniques can be used to effect slidable movement of the guide wire 122 between the retracted and extended positions, but in one embodiment, as shown, the guide wire 122 can extend fully through the first member 112 such that a proximal end 122a extends from the proximal end 112a of the first member to allow a user to grasping and move the guide wire 122 relative to the first member 112. In other embodiments, the guide wire 122 can be coupled to a lever or other device formed on the proximal end 112a of the first member 112 for slidably moving the guide wire 122 relative to the first member 112. A person skilled in the art will appreciate that a variety of techniques can be used to control movement of the guide wire 122 relative to the first member 112.

The first member 112 can also include an indicator or alignment mechanism 124 that is adapted to indicate the direction of movement of the guide wire 122 as it extends from the distal end 112. In particular, as shown in FIG. 2A, an alignment mechanism 124 can be formed on the handle 116a of the first member 112 at a location that is in alignment with the side opening 125 formed in the distal end 112b of the first member 112. Thus, in use, when the first member 112 is inserted into tissue, the alignment mechanism 124 can be used to position the side opening 125 of the first member 112 such that the guide wire 122 will extend toward the distal end 114b of the second member 114, as will be discussed in more detail below.

The second member 114 can also have a variety of configurations, but as indicated above the second member 114 preferably includes a grasping element that is adapted to grasp the guide wire 122 to couple the first and second members 112, 114. While various grasping techniques can be used, in the illustrated exemplary embodiment the grasping element is in the form of a pair of movable jaws 126a, 126b. While not shown, the second member 114 can include an actuating mechanism formed on a proximal end thereof for moving the jaws 126a, 126b between an open and closed position. A person skilled in the art will appreciate that a variety of techniques can be used to move the jaws 126a, 126b between an open and closed position, and that a variety of other grasping mechanisms can be used.

FIG. 2B illustrates the facilitating tool 100 in use. An exemplary method for using the tool 100 is similar to the method previously described with respect to FIG. 1B, however in this embodiment the first member 112 is only inserted through the transverse sinus, and it does not extend toward the second member 114. Rather, once the first member 112 is positioned through the transverse sinus, the guide wire 122 is extended from the distal end 112b of the first member 112. As a result, the guide wire 122 will extend adjacent to the pericardial sac wall toward the oblique sinus. The second member 114 can then be used to grasp the guide wire 122 and pull the first member 112 around the pulmonary veins. As was also previously described with respect to FIG. 1A, an ablation device can then be inserted through the first member 112 to form a lesion around the pulmonary veins, or alternatively the first member 112 can be used to position an ablation device around the pulmonary veins. The later technique can be achieved by, for example, removing the handle 116a on the proximal end 112a of the first member 112 and coupling an ablation device to the first member 112. The first member 112 can then be further pulled to position the ablation device around the pulmonary veins.

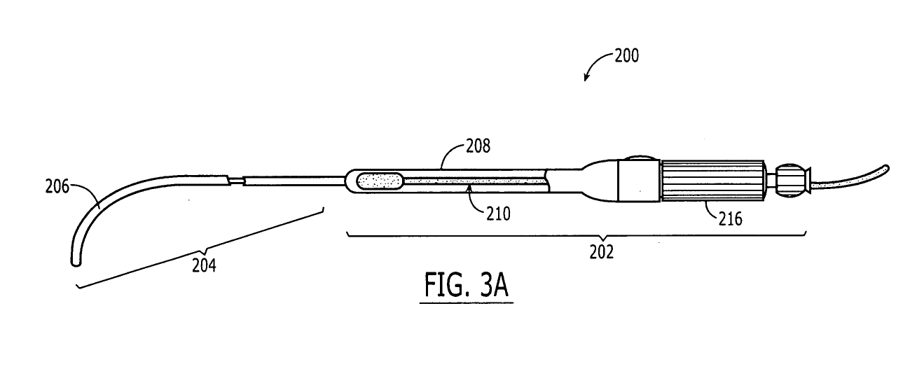

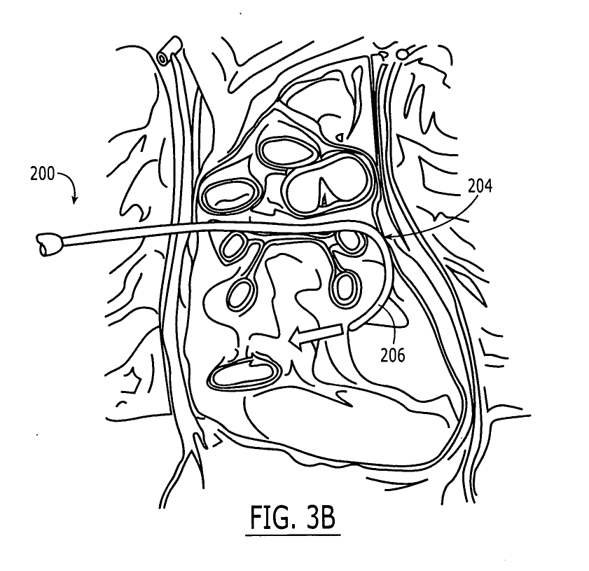

FIGS. 3A and 3B illustrate yet another embodiment of a facilitating tool 200 for use in positioning an ablation device. In this embodiment, the facilitating tool 200 is a single elongate member that is substantially self-guiding. As shown, the tool 200 includes a proximal portion 202 having a handle 216 with an elongate member, e.g., a catheter 208, extending therefrom, and a distal portion 204 extending from the catheter 208 and having a curved tip 204 formed on the terminal end thereof. The catheter 208 extending from the handle 216 can be adapted to receive an ablation element 210 therethrough, as shown, and the curved tip 204 on the distal portion 204 can be adapted to guide the tool 200 around the pulmonary veins to position the catheter 208 and ablation element 210 around the pulmonary veins. A person skilled in the art will appreciate that the catheter 208 of the proximal portion 202 and the distal portion 204 can be a single integral catheter. Moreover, the proximal portion 202 can have a variety of other configurations, including the various exemplary configurations disclosed herein. By way of non-limiting example, the proximal portion 202 can merely include a removable handle that, when removed, allows an ablation element to be coupled directly to the distal portion 204 such that the distal portion 204 can be used to pull and position the ablation element around the pulmonary veins.

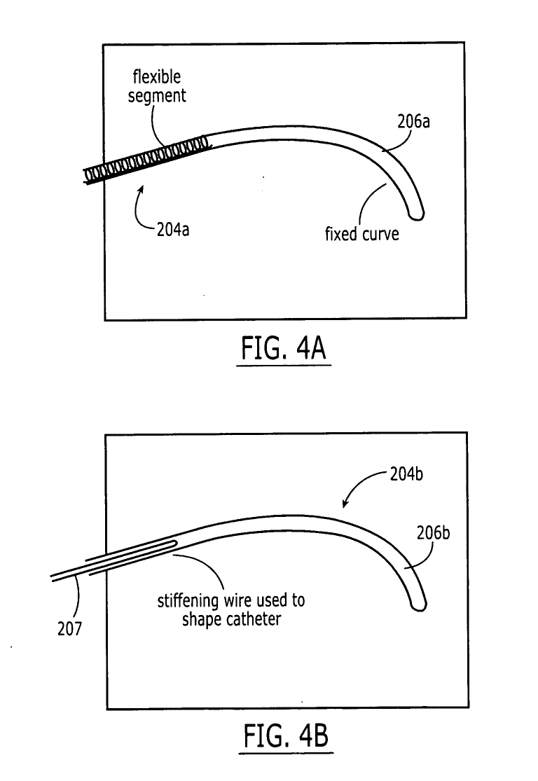

The curved tip 206 on the distal portion 204 can also have a variety of configurations, and the curvature can be formed using a variety of techniques. In an exemplary embodiment, the tip has a shape that is adapted to allow the catheter 208 to be self-guided through the heart and around the pulmonary veins. Since this can depend on the size of the patient, the facilitating tool 200 can optionally be provided with several removable tips, each have a different curvature to allow the surgeon to select the appropriate tip. FIGS. 4A and 4B illustrate two exemplary embodiments of techniques for providing a curved distal tip. In FIG. 4A, the distal portion 204a is substantially flexible except for the curved tip 206, which is substantially rigid such that the tip 206 has a fixed shape. In FIG. 4B, the distal portion 204b includes a stiffening wire 207 disposed therein and adapted to control the shape of the curved tip 206. A person skilled in the art will appreciate that a variety of techniques can be used to form a curved tip 206.

FIG. 3B illustrates facilitating tool 200 in use. The procedure is similar to the procedure described with respect to FIG. 1B, however in this embodiment the tool 200 can be positioned completely around the pulmonary veins without the use of a second device. As shown, the tool 200 is inserted through the transverse sinus, and when the curved tip 206 comes into contact with the pericardial sac wall the tip 206 will extend in an inferior direction toward the oblique sinus. Further movement of the tool 200 will cause the distal portion 204 to pass through the oblique sinus, thereby encircling the pulmonary veins with the tool 200. The distal portion of the tool 204 can then be pulled to position the catheter 208 and ablation element 210 around the pulmonary veins, thereby allowing an ablative lesion to be formed. Alternatively, the tool 200 can be coupled to an ablation device and used to pull and position the ablation device around the pulmonary veins.

A person skilled in the art will appreciate that the various techniques disclosed herein for positioning a facilitating tool around the pulmonary veins can be used to directly position an ablation device around the pulmonary veins. For example, the ablation device can include a distal end that is adapted to couple to a distal end of a catheter, thus allowing the catheter to be used to pull the ablation device around the pulmonary veins. In another embodiment, the distal end of the ablation device can include a guide wire extending therefrom or some other feature to facilitate grasping of the ablation device with a grasping element. The grasping element can then be used to pull the ablation device around the pulmonary veins.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for positioning a device around the pulmonary veins of a heart, comprising:
guiding a first elongate member through the transverse sinus;
guiding a second elongate member through the oblique sinus;
connecting a distal end of the first elongate member to a distal end of the second elongate member; and
removing one of the first and second elongate members to position the other of the first and second elongate members around the pulmonary veins.

2. The method of claim 1, wherein the step of guiding the first elongate member through the transverse sinus further comprises positioning the first elongate member superior to the right superior pulmonary vein and the left superior pulmonary vein, and wherein the step of guiding the second elongate member through the oblique sinus further comprises positioning the second elongate member inferior to the right inferior pulmonary vein and the left inferior pulmonary vein.

3. The method of claim 2, wherein the step of guiding the first elongate member through the transverse sinus further comprises introducing the first elongate member between the superior vena cava and the right superior pulmonary vein, and wherein the step of guiding the second elongate member through the oblique sinus further comprises introducing the second elongate member between the inferior vena cava and the right inferior pulmonary vein.

4. The method of claim 3, wherein the step of guiding the first elongate member through the transverse sinus further comprises positioning a distal end of the first elongate member adjacent to the pericardial sac, and wherein the step of guiding the second elongate member through the oblique sinus further comprises positioning a distal end of the second elongate member adjacent to the pericardial sac.

5. The method of claim 1, wherein the method further comprises the step of inserting an ablation instrument through the first elongate member, and activating the ablation instrument to deliver ablative energy to form a lesion encircling the pulmonary veins.

6. The method of claim 5, wherein the step of activating the ablation instrument further comprises sliding the ablation instrument through the first elongate member to form an extended lesion encircling the pulmonary veins.

7. The method of claim 5, wherein the step of activating the ablation instrument further comprises delivering ablative energy as a beam of energy directed towards epicardial tissue.

8. The method of claim 1, wherein the step of connecting a distal end of the first elongate member to a distal end of the second elongate member further comprises engaging a guidewire extending from the distal end of the first elongate member with a grasping mechanism formed on a distal end of the second elongate member.

9. The method of claim 1, wherein at least one of the first and second elongate members includes optics disposed within a distal end thereof.

10. A method for treating atrial fibrillation, comprising:
guiding a elongate member through the transverse sinus superior to the right superior pulmonary vein and the left superior pulmonary vein of a patient's heart, the elongate member having a distal tip that is adapted to guide the elongate member along the pericardial sac wall and through the pericardial reflection inferior to the left inferior pulmonary vein and the right inferior pulmonary vein to extend out of the patient's heart such that the elongate member encircles the pulmonary veins; and
delivering ablative energy to form a lesion encircling the pulmonary veins.

11. The method of claim 10, wherein the step of guiding the elongate member further comprises forming an incision in the pericardial reflection between the superior vena cava and the right superior pulmonary vein, and inserting the elongate member through the incision to extend out of the patient's heart.

12. The method of claim 10, further comprises forming an incision in the pericardial reflection between the inferior vena cava and the right inferior pulmonary vein, and wherein the elongate member exits through the incision.

13. The method of claim 10, wherein the step of delivering ablative energy further comprises inserting an ablation instrument through the elongate member, and activating the ablation instrument to deliver ablative energy.

14. The method of claim 13, further comprising sliding the ablation instrument through the elongate member to form an extended lesion.

15. The method of claim 10, further comprising, prior to the step of delivering ablative energy, coupling an ablation instrument to a proximal end of the elongate member and pulling the elongate member to position the ablation element around the pulmonary veins.

16. A kit for treating atrial fibrillation, comprising:
at least one elongate member having a distal portion that is adapted to bluntly guide the elongate member through the transverse sinus, along the pericardial sac wall, and through the oblique sinus to encircle the pulmonary veins; and
an ablation instrument adapted to couple to an elongate member and to deliver ablative energy to form a lesion encircling the pulmonary veins.

17. The kit of claim 16, wherein the distal portion of the elongate member is curved.

18. The kit of claim 16, wherein the at least a portion of the elongate member is flexible.

19. The kit of claim 16, wherein the elongate member includes a wire extending therethrough and adapted to provide stiffness and shape to the distal portion.

20. The kit of claim 16, wherein the ablation instrument is slidably disposable through the elongate member.

21. The kit of claim 16, wherein a distal end of the ablation instrument is coupled to a proximal end of the elongate member.

22. The kit of claim 16, further comprising a plurality of elongate members with distal portions that differ relative to one another.

23. The kit of claim 16, further comprising a tool having a grasping element formed on a distal end thereof and adapted to grasp a distal end of the elongate member to pull the elongate member through the oblique sinus.

24. The kit of claim 16, wherein the elongate member further includes a guide wire extending from the distal portion and adapted to guide the elongate member around the pulmonary veins.
